(19) United States
(12) Patent Application Publication
(10) Pub. No.: US 2005/0143366 A1
(43) Pub. Date: Jun. 30, 2005

(54) Compounds for diagnosis, treatment and prevention of bone injury and metabolic disorders

(76) Inventors: William M. Pierce JR., Louisville, KY (US); Leonard C. Waite, Corydon, IN (US); K. Grant Taylor, Louisville, KY (US)

Correspondence Address: STITES & HARBISON, PLLC, 400 W MARKET ST, SUITE 1800, LOUISVILLE, KY, 40202-3352, US

(21) Appl. No.: 11/022,024

(22) Filed: Dec. 22, 2004

Related U.S. Application Data
(60) Provisional application No. 60/532,343, filed on Dec. 24, 2003.

(51) Int. Cl. A61K 31/56; C07J 1/00

(52) U.S. Cl. 514/182; 552/626

(57) ABSTRACT

The present invention relates to compounds of the formula 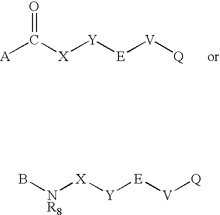 or pharmaceutically acceptable salts thereof useful for the prophylaxis and treatment of degenerative bone disorders and for the acceleration of bone healing.

### CROSS REFERENCE TO RELATED APPLICATIONS

This application claims priority from U.S. Provisional Application Ser. No. 60/532,343 filed Dec. 24, 2003, the entire disclosure of which is incorporated herein by this reference.

### FIELD OF THE INVENTION

This present application relates to compounds for the diagnosis, treatment and prevention of bone injury and metabolic disorders, for example, the promotion of bone healing and the treatment and prophylaxis of degenerative bone disorders.

### BACKGROUND OF THE INVENTION

Bone is a dynamic tissue, consisting of cells in a protein matrix, upon which is superimposed a crystalline structure of various calcium salts. In addition to serving as a rigid support for the body of an animal, bone is an organ which responds to hormones. As a normal regulatory function, in response to interactions with certain hormones, bone cells can solubilize the calcium salts in bone for use elsewhere in the body. However, if this bone resorption is excessive, it can lead to bone degeneration diseases, including Paget's disease of bone and osteoporosis.

Clinical osteoporosis is found in approximately 25% of postmenopausal women, and subclinical osteoporosis, which is responsible for untold numbers of bone fractures in elderly women., is far more widespread. Despite the widespread nature of the bone degeneration diseases, the mechanisms by which they operate are not well understood. In addition, the treatments available are often unsuccessful.

The breakdown of bone by bone cells has been extensively studied, but the specific mechanisms are not clearly defined. One likely scenario is that resorption is caused by the secretion, by bone cells, of acid and proteolytic enzymes. For these enzymes to have their effect, it is likely that the tissue must be decalcified first. Thus, the initiating step is thought to be the acidification of the internal environment of bone, which is responsible for decalcification. Carbonic acid is one of the acids that may facilitate these processes and has so been implicated for many years.

Assuming carbonic acid, which is generated by the enzyme carbonic anhydrase, is involved in bone resorption, then administration of a drug which inhibits carbonic anhydrase should inhibit the liberation of calcium from bone in response to parathyroid hormone (PTH). This is indeed the case, as is first demonstrated in mammals by Waite, et al. in the publication entitled, “Inhibition of Bone Resportion by Acetazolamide in the Rat”, Endocrinology, 87: 11 29 (1970), which studied the effects of the carbonic anhydrase inhibitor Acetazolamide.

Later work in tissue culture showed that inhibition of PTH-induced resorption by acetazolamide is due to a direct interaction at the level of bone (“Carbonic Anhydrous and Bone Remodeling: Sulfonamide Inhibition of Bone Resorption in Organ Culture”, Minkin and Jennings, Science, (June 1970), which is incorporated herein by this reference).

These studies suggest that acetazolamide would be useful as an inhibitor of bone resorption. However, when one administers acetazolamide or other heterocyclic sulfonamide carbonic anhydrase inhibitors to normal animals, no change in plasma calcium concentration is observed. This counterintuitive result is the product of competing effects of acetazolamide. Specifically, in addition to inhibiting calcium dissolution from bone due to PTH response, acetazolamide also causes a systemic acidosis, which itself increases the shift of mineral from bone to blood. These two competing effects mask one another. (See, “Acidosis Inhibits the Hypocalcemic Effect of Acetazolamide”, Lineberry and Waite, Pharmacol. Exp. Ther., 211: 452 (1979), which is incorporated herein by this reference).

The effects of certain sulfonamides on bone resportion have been studied. In this regard, the following has been observed: heterocyclic sulfonamides, such as acetazolamide, which inhibit carbonic anhydrase also inhibit bone resorption; heterocyclic sulfonamides which do not inhibit carbonic anhydrase, do not inhibit bone resorption; the sulfonamides also inhibit the bone resorptive effects of large doses of vitamin D; and since other parameters of bone metabolism are not affected by the sulfonamides, the effects are not the result of a simple toxicity to bone cells.

In U.S. Pat. No. 5,641,762 to Pierce, et al., it is disclosed that osteostats consisting of a bone seeking agent and a carbonic anhydrase inhibitor would be useful in the prophylaxis and treatment of degenerative bone disorders. The disclosure is limited to the combination of sulfonamide and imidazole as carbonic anhydrase inhibitors and tetracycline and diphosphonates as bone seeking agents. The '762 patent also described Mithramycin as a useful reagent for inhibiting bone resorption and provided examples, which were limited to tetracycline internally active acetazolamide (TIA), tetracycline internally active ethoxzolamide (TIE), tetracycline active acetazolamide, tetracycline active ethoxzolamide Δ-1, tetracycline active ethoxzolamide Δ-2, and aminohexyldiphosphonate active acetazolamide.

There remains a need in the art for compounds, pharmaceutically acceptable compositions thereof, methods for use there of, and methods for preparation thereof, which compounds may be used for the diagnosis, prophylaxis and treatment of bone injuries and disorders.

### SUMMARY OF THE INVENTION

The present invention includes a group of compounds, and pharmaceutically acceptable compositions thereof, for the diagnosis, treatment and prevention of bone injury, including injury resulting from bone fracture or from degenerative bone disorders, such as osteoporosis. The present invention also includes a method for use of and a method for the synthesis of the compounds. The present invention also includes methods for diagnosing, treating and preventing bone injure and metabolic disorders. The present invention also includes methods for inhibiting bone resorption. The present invention also includes methods for promoting bone formation.

The compounds of the present invention generally includes three portions: (1) a bone targeting moiety having an affinity for the extracellular inorganic matrix of bone, (2) a bone active moiety having the ability to interact with bone and affect bone metabolism, and (3) a bridging group linking the first and second moieties. The compounds of the present invention may be represented by the following formulas: 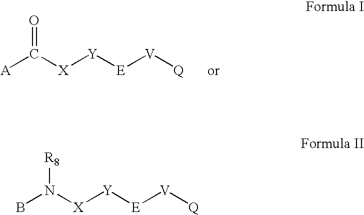
or pharmaceutically acceptable salts thereof, wherein the bone targeting moiety is represented by A and B, the bone active moiety is represented by Q, and the bridging group is represented by the remaining portions.

Bone Targeting Moiety

The bone targeting moiety of the compound is one having an affinity for the extracellular inorganic matrix of bone; for example, the bone targeting moiety could be one of the following: 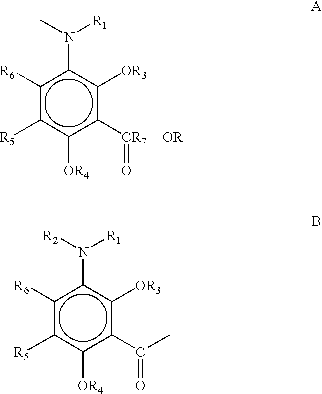
wherein
R₁ is hydrogen, lower alkyl or aryl lower alkyl;
R₂ is hydrogen, lower alkyl or aryl lower alkyl;
R₃ is hydrogen or lower alkyl;
R₄ is hydrogen, aryl lower alkyl, aryl or lower alkyl;
R₅ and R₆ are independently hydrogen or lower alkyl or R₅ and R₆ taken together with the carbon atoms to which they are bonded form a ring containing up to 10 ring carbon atoms and up to a total of 18 carbon atoms;
R₇ is hydroxy, lower alkoxy or NR₈R₉ and
R₈ and R₉ are independently hydrogen or lower alkyl.

Bridging Group

The bridging group of the compound is represented by: —(C═O)—X—Y-E-V— or —(NR₈)—X—Y-E-V—, as mentioned above, wherein:
R₈ is independently hydrogen or lower alkyl;
X is an alkylene group containing from 1-10 carbon atoms on the main chain and up to a total of 20 carbon atoms;

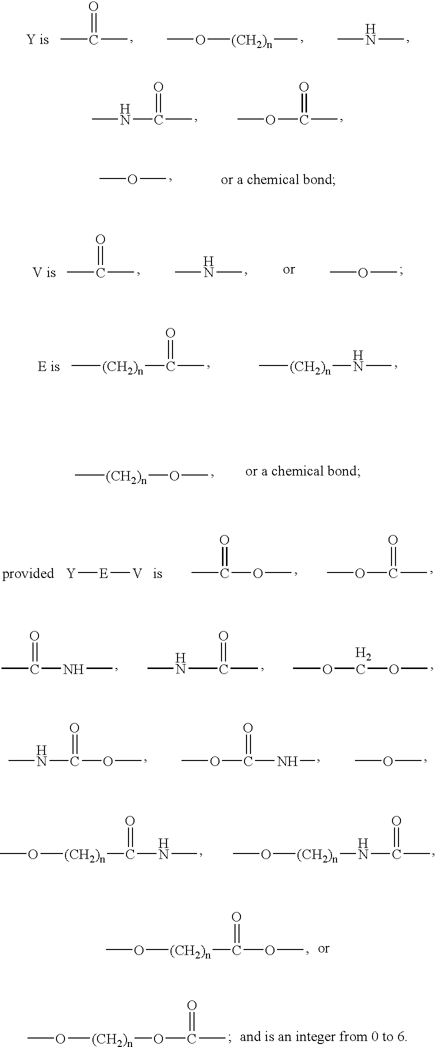

Bone Active Moiety

The bone active moiety, Q, of the compound of the present invention is one which interacts with the bone and affects its metabolism by inhibiting bone resorption, increasing bone formation, or both.

There is a plurality of contemplated agents that may be used as the bone active moiety of the compound of the present invention, for example, specific steroids and related compounds, especially androgens, estrogens, and DHEA (3β-hydroxyl-5-androsten-17-one). For other examples, Vitamin D metabolites and analogs, anti-cancer agents, proton pump inhibitors, non-steroidal anti-inflammatory (NSAIDs) hormones, free radical scavengers, growth factors, autocoids, parathyroid hormone, RANK-L, cathepsin inhibitors, ipriflavone, matrix metalloproteinase inhibitors, HMG CoA reductase inhibitors, NO generating agents, and carbonic anhydrase inhibitors, such as acetazolamide, etholazolamide, methazolamide, benzolamide, and the like, such as those described in U.S. Pat. Nos. 5,055,480; 5,059,613; 5,242,937 and 5,641,762, each of which is incorporated herein by this reference.

Regardless of which agent is used, it is present in a form which is less a hydroxy group or an amino group or oxo group, whichever is present, wherein the portion of the bridging group, V, is bonded to the carbon atom devoid of said hydroxy group, oxo group or amino group.

As used herein, the term “lower alkyl”, when used alone or in combination, refers to alkyl groups containing 1 to about 6 carbon atoms. They may be straight-chained or branched. Examples includes methyl, ethyl, propyl, isopropyl, n-butyl, sec-butyl, t-butyl, isobutyl, n-pentyl, isopentyl, neopentyl, n-hexyl, and the like.

The term “alkylene”, as used herein, is a bridging group that contains 1 to about 10 carbon atoms in the main chain, which, for purposes of this application, is preferably a straight chain. However, the main chain may be branched (i.e., they are alkyl-substituted on the main chain). As used herein, the total number of carbon atoms on the alkylene chain, including the main chain and branched substituents, range from 1 to about 20 carbon atoms. In addition, the alkylene group may be substituted with other groups such as hydroxy, amino, lower alkyl amino or di-lower alkyl amino.

“Aryl”, when used alone or in combination with other groups, refers to an aromatic group containing only ring carbon atoms and having about 6-14 ring carbon atoms and up to a total of about 18 carbon atoms. Examples include phenyl, α-naphthyl, β-naphthyl, tolyl, xylyl, and the like.

“Aryl lower alkyl” refers to an aryl group bonded to a bridging alkyl group, as defined herein. Examples include benzyl, phenethyl, naphthylethyl, and the like.

“Lower alkoxy” refers to any of the above mentioned alkyl or aryl groups linked to an oxygen atom.

Each of the aforementioned substituents could be substituted or un substituted. For example, “lower alkyl” may include substituted lower alkyl.

As will be apparent upon reading this specification, in addition to the compounds described herein, the present invention is also directed to the pharmaceutical compositions containing a pharmaceutically effective amount of the compounds, a method for the sythesis of the compounds, and a method for the use of the compounds for the treatment of bone disorders or for acceleration of healing in an animal, especially mammals, including cats, dogs, horses, rabbits, rats and humans, comprising administering to the animal in need of such treatment a pharmaceutically effective amount of the compounds.

### DESCRIPTION OF THE DRAWINGS

FIG. 1 is a bar graph showing the distal femur strength of animals including those treated with an exemplary compound of the present invention.

### DETAILED DESCRIPTION OF THE INVENTION

The present invention is a compound for the treatment and prevention of bone injury, including injury resulting from bone fracture or from degenerative bone disorders, such as osteoporosis. Additionally, the present invention includes a method for use of and a method for the synthesis of the compound.

The compound of the present invention generally includes three portions: (1) a bone targeting moiety having an affinity for the extracellular inorganic matrix of bone, (2) a bone active moiety having the ability to interact with bone and affect bone metabolism, and (3) a bridging group linking the first and second moieties. The compound of the present invention may be represented by the following formulas: 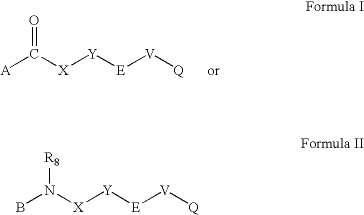
or pharmaceutically acceptable salts thereof wherein the bone targeting moiety is represented by A and B, the bone active moiety is represented by Q, and the bridging group is represented by the remaining portions.

Bone Targeting Moiety

The bone targeting moiety of the compound is one having an affinity for the extracellular inorganic matrix of bone; for example, the bone targeting moiety could be one of the following: 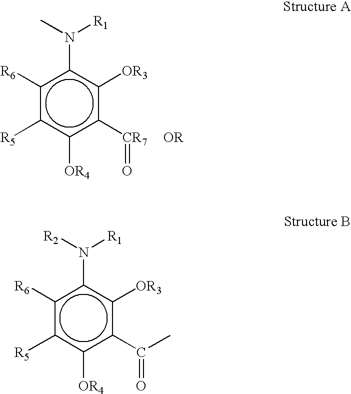
wherein
R₁ is hydrogen, lower alkyl or aryl lower alkyl
R₂ is hydrogen, lower alkyl or aryl lower alkyl;
R₃ is hydrogen or lower alkyl, for example, an alkyl group containing about 1 to 3 carbon atoms, or aryl lower alkyl, such as benzyl;
R₄ is hydrogen, aryl lower alkyl, aryl or lower alkyl;
R₅ and R₆ are independently hydrogen or lower alkyl or R₅ and R₆ taken together with the carbon atoms to which they are bonded form a ring containing about 6 to 14 ring carbon atoms and up to a total of 18 carbon atoms, which formed ring may be monocyclic, bicyclic or tricyclic; R₇ is hydroxy, lower alkoxy or NR₈R₉ and
R₈ and R₉ are independently hydrogen or lower alkyl.

In certain embodiments, the moiety may be bonded to the remainder of the chain at the nitrogen atom attached to R₁ or R₇. In other embodiments, the moiety may be bonded to the remainder of the chain as shown in Structure A.

Bridging Group

The bridging group of the compound is represented by —(C═O)—X—Y-E-V— or —(NR₈)—X—Y-E-V—, and is described above. As defined herein, X is an alkylene chain containing up to about 10 carbon atoms in the main chain and up to a total of about 20 carbon atoms. In certain embodiments, X may contain a total of 1 to about 6 carbon atoms. The alkylene chain may be a straight chain or it may contain branches.

It is to be understood that YEV is to be read from left to right, i.e., it is the last atom on the right (V) in the definition of YEV which is bonded to the compound's bone active moiety, Q. Thus, for example, when YEV is 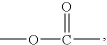
it is to be understood that, as written, the 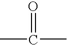
and not the O is bonded to Q.

Bone Active Moiety

The bone active moiety, Q, of the compound of the present invention is one which interacts with the bone and affects its metabolism by inhibiting bone resorption, promoting bone formation, or both.

As mentioned above, there are a plurality of contemplated agents that may be used as the bone active moiety of the compound of the present invention. Examples of such agents which inhibit bone resorption include: Androgenic agents; Carbonic anhydrase inhibitors; Cathepsin inhibitors; DHEA (3β-hydroxyl-5-androsten-17-one); Estrogenic agents; Free radical scavengers; HMG CoA reductase inhibitors (statins); Ipriflavone; Matrix metalloproteinase inhibitors; NO generating agents (blood flow); Non-steroidal anti-inflammatory agents (NSAIDs); Proton pump inhibitors; Sex hormones, preferably in their steroid form; and Vitamin D metabolites and analogs. Examples of such agents which promote bone formation include: Growth factors; Autocoids; Estrogenic agents; Parathyroid hormone; and RANK-L.

Examples of specific carbonic anhydrase inhibitors, useful in the present invention, include: acetazolamide, etholazolamide, methazolamide, benzolamide, and the inhibitors described in U.S. Pat. Nos. 5,055,480; 5,059,613; 5,242,937 and 5,641,762, each of which is incorporated herein by this reference.

Examples of specific Vitamin D metabolites and analogs, useful in the present invention, include: ergocalciferol, (vitamin D2), cholecalciferol, (vitiamin D3), 25-hydroxy-ergocalciferol, 1,25-dihydroxyergocalciferol, 25-hydroxy-cholecalciferol, 1,25-dihydroxycholecaciferol, 24,25-dihydroxy vitamin D3.

With regard to the agents which are steroids, by definition, a steroid consists of four ring structures, an A ring, B ring, C ring, and D ring. The D ring, which is a cyclopentyl ring, contains a C═O or C—OH at C-17 of the steroid. In the present invention, the substituent at C-17 (hydroxy or oxo group) may be derivatized, as defined herein, and for purposes of the specification. When the bone active moiety, Q, is a steroid, it will exclude hydroxy and oxo-substituent at C-17. As will be described further below, the substituent on C-17 is encompassed by the YEV variable. Thus, when Q is a steroid, it is defined as the steroid having these substituents at C-17. Thus, in an embodiment of the present invention, V of the bridging group is bonded to C-17 of the steroid.

Examples of estrogens useful in the present invention are estradiol, estrone, estriol, xenoestrogens, phytoestrogens, and the like. Examples of androgens, useful in the present invention include testosterone, 5α-dihydrotestosterone, androstenedione, etiocholanolone, epiandrosterone, androsterone, 17α-methyl testosterone, fluoxymesterone, 17α-ethyl testosterone, 17α-methylandrostan-3β,17 β-diol, androstan-3a, 17 β-diol, androstan-3α-17 α-diol, androstan-17β-o13-one, androstane-17α-o1-3 one, Δ⁵-androsten-3α, 17β-diol, Δ⁵-androstene-3β,17β-diol, Androstane-3-17-dione, Δ⁴-androstenedione, and the like.

Regardless of whether the agent used as the bone active moiety is a steroid or not, it is less a hydroxy group, an amino group, or an oxo group, whichever is present, wherein the portion of the bridging group, V, is bonded to the carbon atom devoid of said hydroxy group, oxo group, or amino group. The V moiety of the bridging group forms the ether, amide or ester, which permits the bridging group to bind to Q. The V moiety is thus attached to Q at the position wherein the oxo group, amino group or hydroxy group was originally present. Thus, if Q is less an amino group then Q is bonded to the bridging group as an amide and YEV is

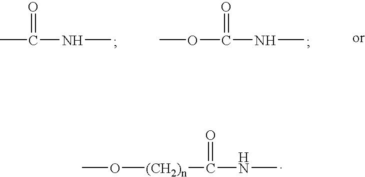

On the other hand, if Q is less the oxo group, this means that the oxo group originally present on, for example, DHEA or the sex hormone, has been reduced by reagents known in the art, such as H₂/Pd, NaBH₄, LiAlH₄, amalgamated zinc and HCl, or hydrazine and a base such as KOH or potassium tert-butoxide to form the corresponding alcohol. The alcohol thus formed may be oxidized to the corresponding carboxylic acid by reagents known in the art, (e.g., aqueous permanganate or chromic oxide, and the like) and then reacted with a bridging group having an alcohol or amine moiety thereon under conditions known in the art to form an ester or amide.

Alternatively, the hydroxy group may react with a bridging group having an acylating moiety to form an ester or the hydroxy group may react with a bridging group under conditions known in the art to form an ether. If, on the other hand, Q is less the hydroxyl group, then Q is bonded to the bridging group as an ester 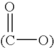
or ether, or, if the hydroxy group is oxidized to form the acid, as a different ester 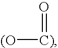
or even an amide 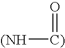
if the bridging moiety YE has a free amino group. Thus, if Q is less an oxo or hydroxy group, then Q is bonded to the bridging group as an ester or ether or amide and YEV is

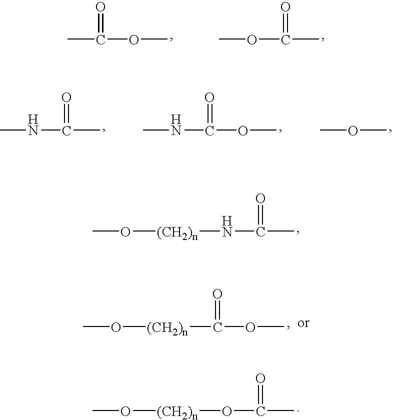

Synthesis of the Compounds of the Present Invention

The compounds of the present invention are prepared by techniques recognized in the art. For example, the compounds of the present invention may be prepared by the schemes set forth below.

With respect to compounds of Formula I, the following is exemplary:

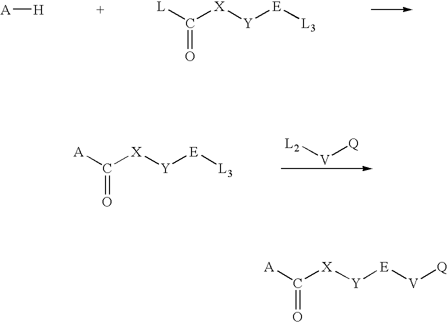

Likewise, with respect to compounds of Formula II, the following is exemplary:

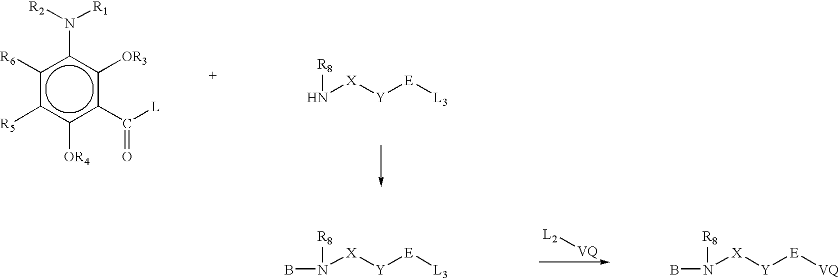

As defined herein, AH, which contains an NR₁ group, reacts with 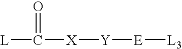
under amide-forming conditions to form the corresponding amide, wherein L is a leaving group, such as hydroxy, lower alkoxy, halogen, and the like and L₃ is hydrogen when Y-E ends with an NH or O and is halide or OR₁₁ wherein R₁₁ is hydrogen or lower alkyl when YE ends with acyl group, or is halide, or brosylate, tosylate or mesylate or hydrogen when YE ends with a methylene (CH₂) group. The reaction may be conducted in a solvent, which does not react with the reactants or products and in which the reactants are soluble. The solvent may be volatile.

The reaction is conducted at effective temperatures, for example, the reaction may be performed at room temperature up to the refluxing temperature of the solvent. The reaction is conducted for sufficient time to form the desired product.

The product thereof is 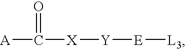
which has at the other end, (i.e., the YE end), a functional group that is reacted with the L₂V-Q to form the compound of Formula I under acylating conditions, wherein L₂ is hydrogen when V is O or NH or L₂ is OR₁₀ or halide when V is 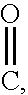
and R₁₀ is hydrogen or lower alkyl.

For example, if VL₂ contains an hydroxy or amino, the hydroxy group or amino may react with the acyl group on YE. For instance, if E were a chemical bond, and Y were an acid or acylating derivative thereof, the reaction of HVQ with 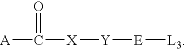
would form an ester or amide. On the other hand, L₂VQ may be an acylating derivative, which may be converted to an amide or ester group when it reacts with the terminal OH or amino group on YEL₃ to form an ester or amide, respectively.

Again, as indicated above, the acylating reaction may be conducted in a solvent, which does not readily react with either the products or the reactant and in which the reactants are readily soluble. Preferably, the solvent is volatile so that it can easily be removed by evaporation. The reaction is performed at effective temperatures, which may range from room temperature up to the reflux temperature of the solvent, and is conducted for sufficient time to form the desired product.

If an ether is formed, then LV is OH and YE ends with a methylene group. Under those conditions, the reaction is conducted under Williams conditions, wherein QOH is converted to the corresponding QO^{θ} and is reacted with 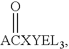
wherein L₃ is halide or other good leaving group, and YE ends with a CH₂ group. Alternatively, the QOH and 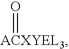
wherein L₃ is a halide that may be reacted directly with a hydroxide salt, such as KOH or NaOH, in dimethyl sulfoxide.

Alternatively, if QVL₂ contains a hydroxy group at position 17 of the steroid and if XYE is 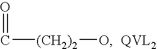
may be reacted with a cyclic anhydride, such as succinic anhydride, a mixed anhydride or an acyl halide, the product of which reacts with AH to form the product of Formula I.

The reaction to form Formula II is similar to the reactions described above except that B contains the acyl group, so that BL is reacted with the amine HR₈XYE-L₃ under amide forming conditions to form BN(R₈)XYE L₃, which in turn is reacted with L₂VQ to form the product of Formula II. It is to be noted that L₁, L₂ and L₃, R₁, R₂, R₃, R₄, R₅, R₆, R₈, XYE, V, and Q are as defined above. Therefore, the comments herein, with respect to the amide forming reactions and with the reactions with L₂QV, are also applicable to these reactions.

Of course, the reactions may be performed in a reverse order. For example, the compound of Formula I may also be formed as follows:

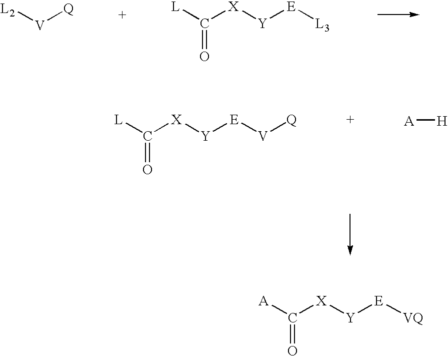

Similarly, the compound of Formula II may be formed by reacting: 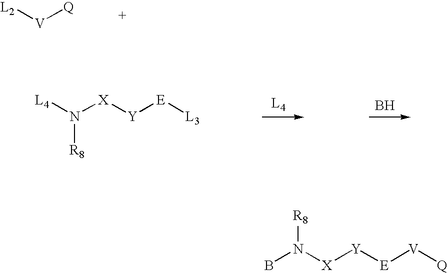
wherein L₄ is hydrogen or, if the amine is reactive with V or E, then it is an amine protecting group known in the art.

If any of the groups on A, B, X, Y, E, V, or Q have groups which are reactive with any of the reagents used or with any of the reactants or products, then they would be protected by protecting groups known in the art to avoid side reactions. Such protecting groups are used in synthetic organic chemistry and are well known in the art. Examples may be found in “PROTECTIVE GROUPS IN ORGANIC SYNTHESIS” by T. W. Greene, John Willey & Sons, Inc., N.Y., 1981, (“Greene”), which is incorporated herein by this reference.

The following illustrations of the synthesis of some of the compounds of the present invention are set forth for exemplary purposes:

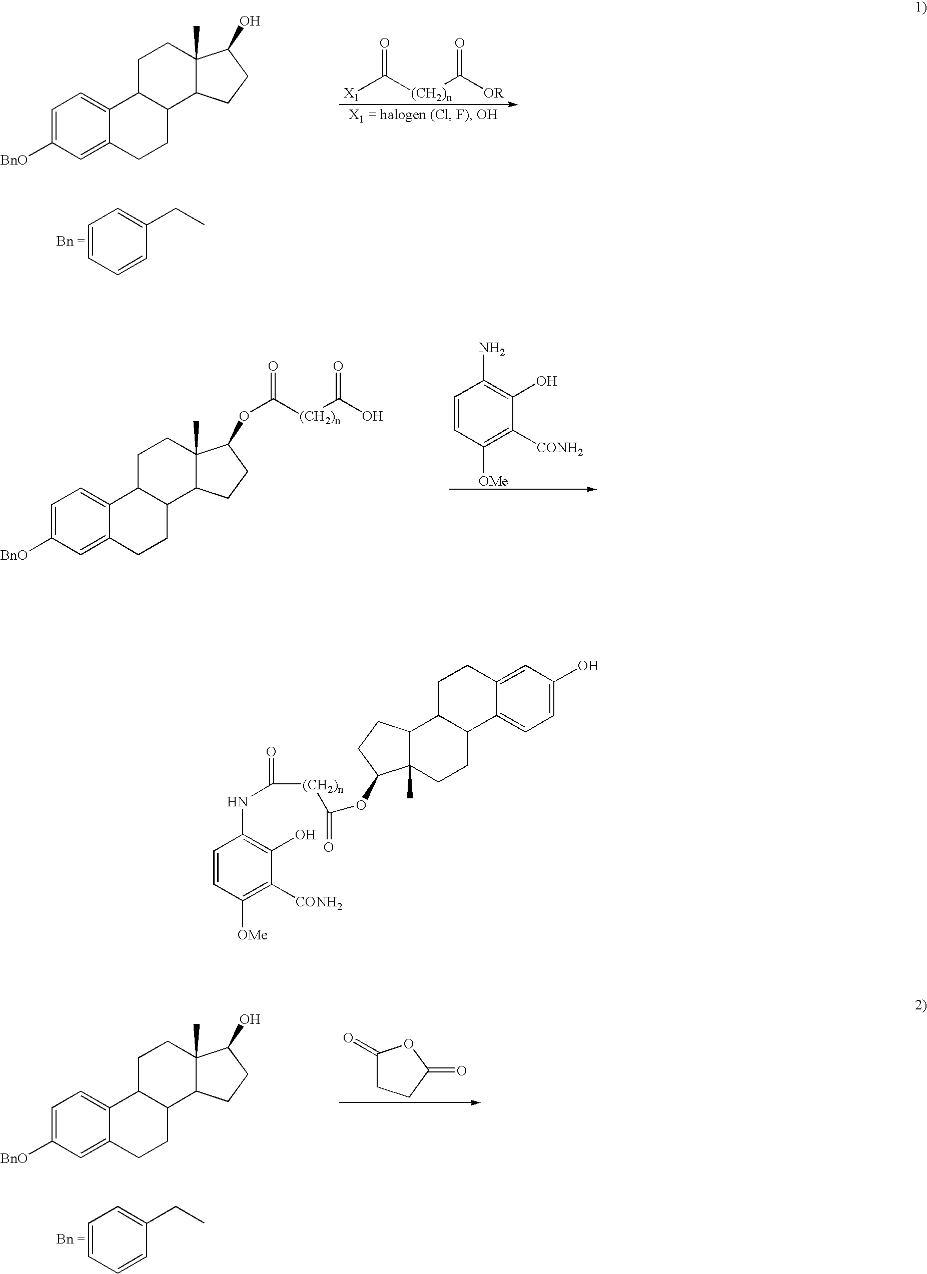

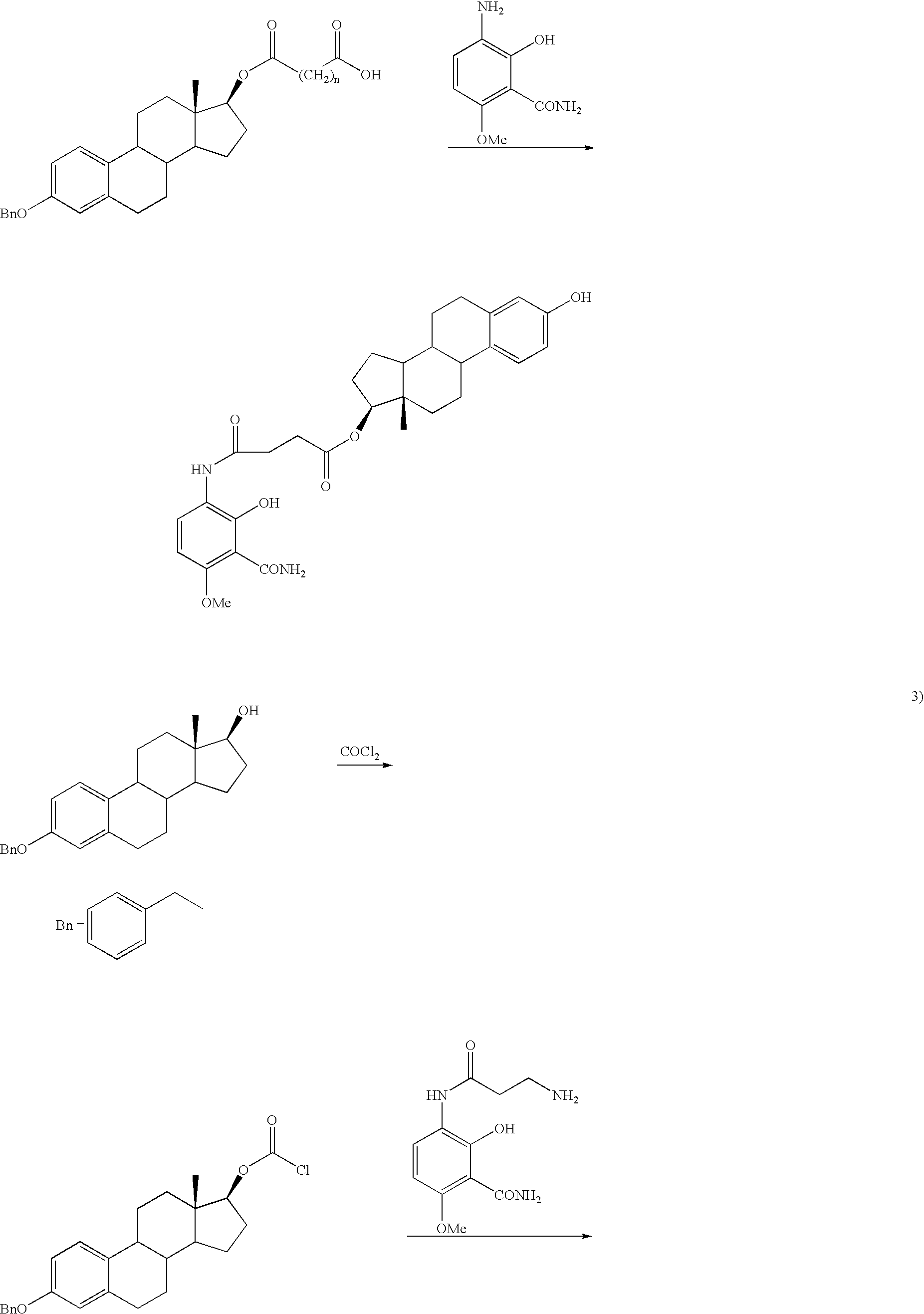

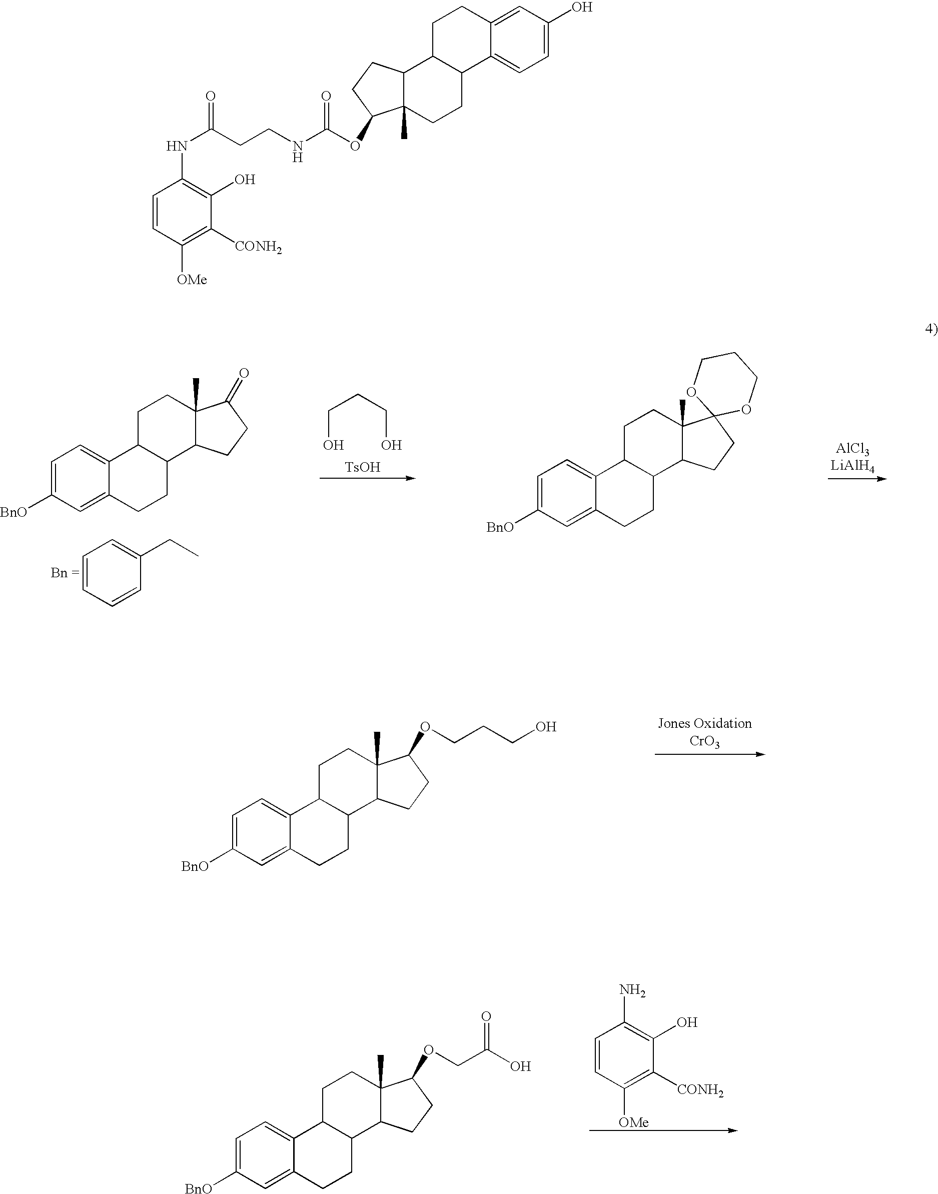

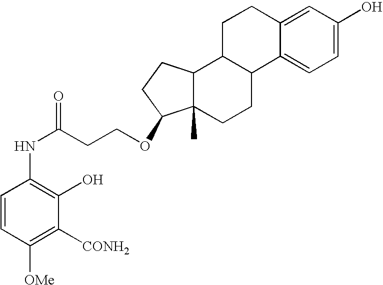

Use and Efficacy of the Compounds of the Present Invention

As described above, the compounds of the present invention are characterized by two active moieties. The bone-targeting moiety (i.e., the benzamide portion of the molecule) having the ability to bind to calcium with a tendency to accumulate in bone and to incorporate into its crystal lattice, and the bone active moiety, which interacts with the bone and affects bone metabolism by inhibiting bone resorption, increasing bone formation, or both. For example, if the second moiety is vitamin D, then its interaction with bone tends to strengthen and increase bone formation. On the other hand, the steroids contemplated by the present invention exhibit bone activity and may both inhibit bone resorption and stimulate bone formation. In addition, the carbonic anhydrase inhibitors contemplated by the present invention inhibit the enzyme carbonic anhydrase which catalyzes the reversible hydration of carbon dioxide to carbonic acid and thus it is an inhibitor of bone resorption. Other bone active agents exert their known effects in a manner relatively specific to bone.

The performance of the compounds of the present invention can be facilitated immediately at the bone site, having the active site of both the benzamide and the bone active moieties available (not a pro-drug). Additionally, the performance of the compounds of the present invention can be facilitated stepwise, first by the bone seeking moiety which affinity localizes the compound at the bone site. Once anchored at the bone site, the other moiety of the molecule, i.e., the bone active domain, interacts with the bone and affects either an increase in bone formation or inhibits bone resorption.

On the other hand, the compounds of the present invention, may be a pro-drug, wherein the active site regarding bone activity is internal in the compound and not immediately available and will exhibit no initial activity; however, when subjected to the enzymatic or hydrolytic conditions occurring at the bone site, the bone active domain will be released. This action reflects an idealized feed-back system and is elicited only in response to the specific need.

Without wishing to be bound by theory or mechanism, it is believed that the compounds of the present invention interact with the calcium in the bone in the following manner, which is described using an exemplary embodiment of the present invention:

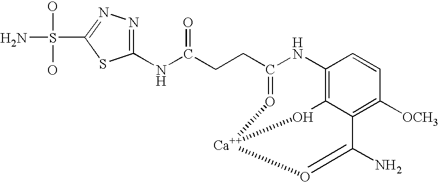

As shown by the example, three positions of the bone targeting moiety interact with the calcium resulting in the compounds of the present invention localizing in the bone. The R₂ moiety, especially the OH, the acyl group of COR₇ moiety, and the acyl group bonded to NHR₁, bind to the calcium of the bone.

The compounds of the present invention may contain one or more asymmetric carbon atoms and may exist in racemic and optically active forms. Depending upon the substituents, the present compounds may form addition salts as well. All of these other forms are contemplated to be within the scope of the present invention.

The present compounds may exist in stereoisomeric forms and the products obtained thus can be mixtures of the isomers.

The active ingredients of the therapeutic compositions and the compounds of the present invention exhibit activity in the treatment and prevention of degenerative bone disorders when administered in effective amounts. These amounts can be determined by a physician. Alternatively, the active ingredients may be administered in amounts ranging from about 0.1 μg to about 100 mg per kilogram of body weight per day. A dosage regimen could be from about 1 μg to about 10 mg per kilogram of body weight per week, and such dosage units may be employed so that a total of from about 7 μg to about 700 mg of the active compound for a subject of about 70 kg of body weight are administered in a 24-hour period.

This dosage regimen may be adjusted to provide the optimum therapeutic response and may be administered from once a day to once a week in dosages of about 50 mg per administration. For example, several divided doses may be administered daily or the dose may be proportionally reduced as indicated by the exigencies of the therapeutic situation. One practical advantage is that the active compound may be administered in a convenient manner such as by the oral, intravenous, intramuscular or subcutaneous routes.

The active compound may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least about 5% of active compound. The percentage of the compositions and preparations may, of course, be varied and may be between about 1 to about 10% of the weight of the unit. The amount of active compound in such therapeutically useful compositions is such that a suitable dosage will be obtained. Preferred compositions or preparations according to the present invention are prepared so that an oral dosage unit form contains between about 5 and 500 mg of active compound.

The tablets, troches, pills, capsules and the like may also contain the following: a binder such as cum tragacanth, acacia, corn starch or gelatin; excipients; disintegrating agents such as corn starch, potato starch, alginic acid and the like; lubricants; and a sweetening agent such as sucrose, lactose or saccharin may be added or a flavoring agent such as peppermint, oil of wintergreen, or cherry flavoring. When the dosage unit form is a capsule, it may contain, in addition to materials of the type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavoring such as cherry or orange flavor. Of course, any material used in preparing any dosage unit form should be pharmaceutically pure and substantially non toxic in the amounts employed. In addition, the active compound may be incorporated into sustained release preparations and formulations.

The active compound may also be administered parenterally or intraperitoneally. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. The form should be sterile and fluid to the extent that easy syringability exists. It should be stable under the conditions of manufacture and storage and should be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants.

The prevention of the action of microorganisms can be brought about by various antibacterial and antifungal agents, for example, parabens, chlorobutanol, phenol, sorbic acid, thimerosal, and the like. In many cases, the inclusion of isotonic agents may be desirable, for example, sugars or sodium chloride. Prolonged absorption of the injectable compositions can be brought about by the use in the compositions of agents, delaying absorption, for example, aluminum monostearate and gelatin.

Sterile injectable solutions may be prepared by incorporating the active compound in the required amount in the appropriate solvent with various of the other ingredients enumerated above, followed by filtered sterilization. Generally, dispersions may be prepared by incorporating the various sterilized active ingredient into a sterile vehicle containing the basic dispersion medium and the required other ingredients from those enumerated above. In the case of sterile powders for the preparation of sterile injectable solutions, methods of preparation include vacuum drying and freeze drying techniques, which yield a powder of the active ingredient plus any additional desired ingredient from previously sterile filtered solution thereof.

As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

Parenteral compositions may be formulated in dosage-unit form for ease of administration and uniformity of dosage. Dosage-unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the dosage-unit forms of the invention may be chosen based upon (a) the unique characteristics of the active material and the particular therapeutic effect to be achieved, and (b) the limitations inherent in the art of compounding such an active material for the treatment of disease in living subjects having a diseased condition in which bodily health is impaired as herein disclosed in detail.

The active ingredient may be compounded for convenient and effective administration in effective amounts with a suitable pharmaceutically acceptable carrier in dosage unit form as hereinbefore disclosed. A unit dosage form can, for example, contain the principle active compound in amounts ranging from, for example, about 0.1 to about 1000 mg or, for another example, from about 5 to about 500 mg. Expressed in proportions, the active compound is generally present in from about 1 to about 100 mg/ml of carrier. In the case of compositions containing supplementary active ingredients, the dosages may be determined by reference to the usual dose and manner of administration of the ingredients.

The following non-limiting examples further illustrate the present invention.

### EXAMPLE 1

The following scheme depicts the synthetic route utilized to prepare the product described in Example 1.

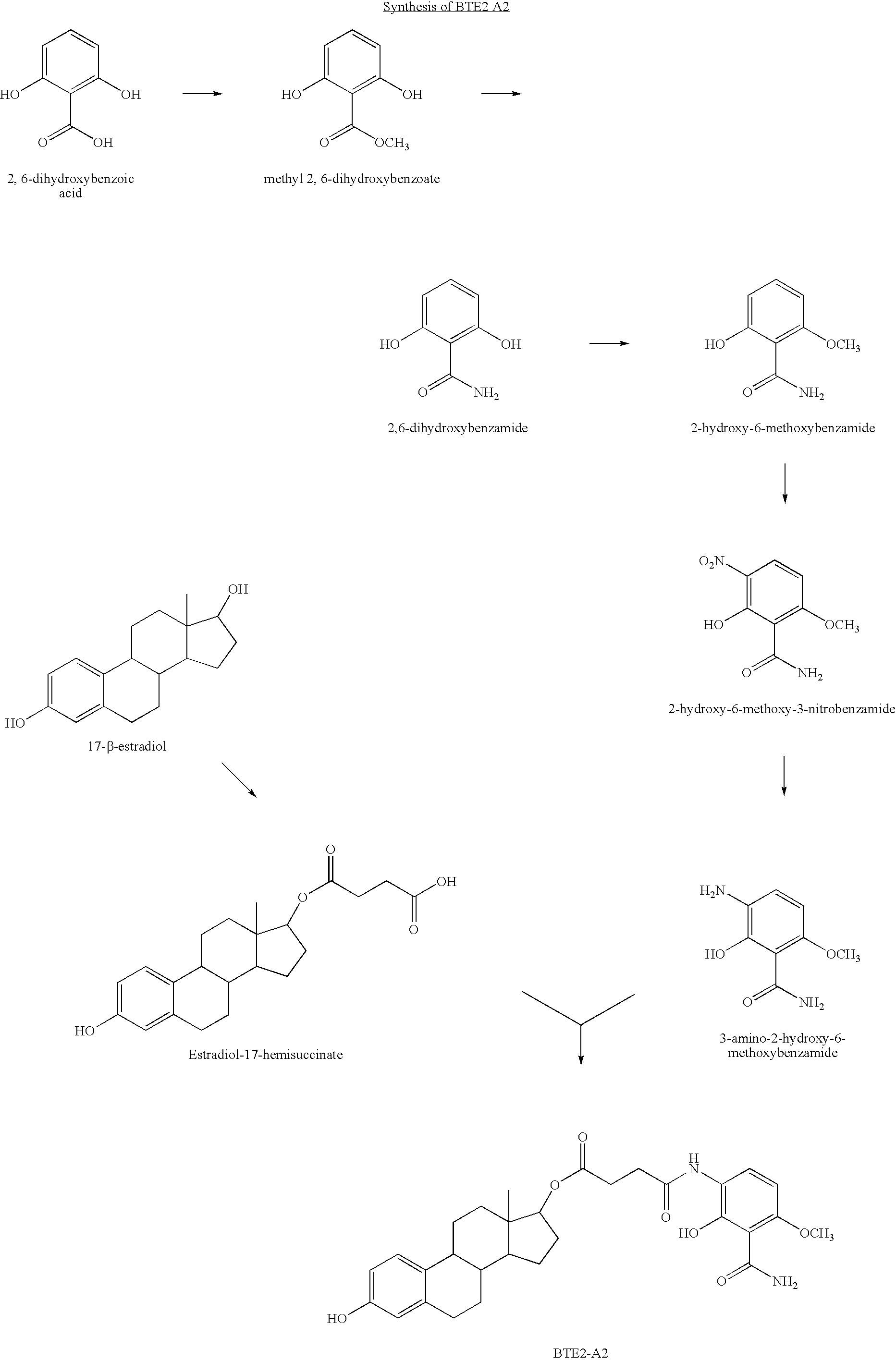

### A. 2,6-Dihydroxybenzoic Acid

2,6-Dihydroxybenzoic acid (CAS [303-07-1] is purchased from Aldrich Chemical Company and is recrystallized from hot water then dried in a vacuum oven before use.

### B. Methyl 2,6-Dihydroxybenzoate

About one hundred grams of 2,6-dihydroxybenzoic acid is dissolved in about 1000 mL of NH₄OH and about 1.1 eq of AgNO₃ is added. About 1.1 eq of CH₃I is added over about 30 minutes at about 0 to 5° C. The mixture is allowed to stir for about 12 hours. Methyl iodide and ammonia are removed under reduced pressure and the sample is recovered by filtration.

### C. 2,6-Dihydroxybenzamide

About one hundred grams of methyl 2,6-dihydroxybenzoate is dissolved in about 1000 mL of NH₄OH and stirred occasionally at room temperature for about 24 hours. Ammonia is removed under reduced pressure and the product crystallizes and is recovered by filtration. The residual water is removed by dissolving in benzene and removing water by azeotropic distillation using a Dean-Stark trap. Benzene is removed under reduced pressure.

¹H NMR (500 MHZ, DMSO d₆) δ 12.64 (s, 2H, 20H and 6-OH), 8.20 (d, 2H, NH₂), 7.16 (t, 1H, J=8.5 Hz, H4, H3 or H5), 6.35 (d, 2H, H3 and H5, J=8.5 Hz, H3 or H5, H4).

¹³C NMR (500 MHZ, DMSO-d₆) δ 172.37 (C=0), 160.70 (C2 and/or C6), 133.63 (C3 and/or C5), 107.09 (C4), 102.39 (C1). DMSO peak calibrated at δ 39.5.

### D. 2-hydroxy-6-methoxyhenzamide

In a 500 mL round-bottomed flask about 16.5 g of 2,6-dihydroxybenzamide is dissolved in 300 mL of dry acetone. To this is added about 32.79 g K₂CO₃ and the resultant product is stirred for about 30 minutes at room temperature. About 12 ml of about 1.5 mM (CH₃)₂SO₄ is added dropwise and the reaction mixture is heated to reflux and held there for about 15 hours. The mixture is cooled in an ice bath and then is filtered.

The filtered solid is washed multiple times with about 50 mL portions of acetone. Acetone is then removed using a rotary evaporator. The residue is mixed with 1N NaOH, then about 300 mL CHCl₃, is added and the mixture is transferred to a separatory funnel. The lower layer is removed and the aqueous phase is further washed multiple times with about 50 mL portions of CHCl₃.

The aqueous layer is acidified to about pH=3-4 with 1N HCl and a white precipitate forms. The precipitate is redissolved in about 300 mL CHCl₃, then is washed multiple times with about 100 mL portions of 1 M NaCl (aq). The chloroform extract is concentrated under rotary evaporation. Finally traces of water and CHCl₃ are removed by azeotropic distillation from a benzene solution. Benzene is stripped under vacuum distillation.

### E. 2-hydroxy-6-methoxy-3-nitrobenzamide

About 8.5 g 2-hydroxy-6-methoxybenzamide is dissolved in about 100 mL of glacial acetic acid in a round bottom flask. This solution is cooled in an ice bath and concentrated HNO₃ (10 mL/0.157 mol) is added in three portions over a period of about 30 minutes. The mixture is stirred for about 6 hours at about 0 to 5° C., then stirred for about 18 additional hours at room temperature.

The reaction mixture was diluted with about 1.0 L of cold water and the resulting solid is recovered by filtration. The solid is washed with about 500 mL of H₂O then multiple portions of about 100 mL ethanol. The product is dried in a vacuum desiccator over P₂O₅.

¹H NMR (500 MHZ, DMSO-d₆) δ 15.72 (s, 1H, 2-OH), 8.42 (d, 2H, C—NH₂), 8:15 (d, 1H, H4, J=8.5 Hz, H4, H5), 6.74 (d, 1H, H5, J=8.5 Hz, H5, H4), 4.00 (s, 3H, OCH₃)

¹³C NMR (500 MHZ, DMSO-d₆) δ 170.44 (C=0), 163.04 (C3), 158.90 (C2), 131.53 (C1), 130.71 (C4), 105.25(C6), 101.86 (C5), 57.14 (—OCH₃).

### F. Synthesis of 3-amino-2-hydroxy-6-methoxybenzamide

About 7.3 g of 2-hydroxy-6-methoxy-3-nitrobenzamide is slurried in about 250 mL of dry CH₃OH, and about 2.5 g of 10% Pd/C catalyst is added. The mixture is placed in a Parr hydrogenator under about 46 p.s.i. of H₂. The mixture is held for about 18 hours during which time the H₂ pressure decreases to about 9.5 p.s.i. The mixture is filtered using Celite and concentrated under vacuum at about 35 to 40°.

¹H NMR (500 MHZ, DMSO-d₆) δ 14.43 (s, 1H, 2-OH), 8.15 (d, 2H, —C NH₂), 6.73 (d, 1H, H5, J=8.5 Fz, FE), H4), 6.33 (d, 1H, H4, J=8.5 Hz, H4, H5), 4.38 (s, 2H, 5-NH₂), 3.79 (S, 3H, —OCH₃).

¹³CNMR (500 MHZ, DMSO-d₆) δ 172.4 1 (C=0), 151.31 (C2), 149.68 (C3), 131.32 (C6), 116.57 (C5), 102.58 (C1), 100.64 (C4), 55.96 (-OCH₃).

### G. Synthesis of Estradiol-17-hemisuccinate

About 10 grams of 17-β-estradiol is dissolved in about 200 mL benzene with about 4 mL pyridine in a round-bottom flask fitted with a Dean-Stark trap. The solution is heated to reflux to remove traces of water. The solution is cooled and about 12 g (excess) of succinic anhydride is added. The mixture is heated to reflux and held for about 24 hours.

The mixture is cooled and filtered, then concentrated using rotary evaporation. About 50 mL CH₃OH is added, along with about 2 g NaHCO₃ and about 10 mL H₂O. The mixture is stirred for about 12 hours and filtered. To the filtrate, about 1N HCl is added until about pH=7, then about 2.0 L of 0.1N HCl (cold) is added. The product is recovered by filtration and is recrystallized from benzene.

### H. Synthesis of BTE2-A2

About 4 grams of estradiol-17-O-hemisuccinate is dissolved in 25 mL dimethylformanide (DMF) and about 1.5 g of N-hydroxybenzotriazole (HOBT) is added. The mixture is cooled to about 0-5° C. and is stirred for about 30 minutes. Diisopropylcarbodiimide (about 1.34 g) is added, followed by stirring for about 30 minutes at about 0 to 5° C. A solution of about 1.955 g of 3-amino-2-hydroxy-6-methoxy-benzamide in about 10 mL DMF is added and the mixture is stirred for about 24 hours at room temperature. The reaction is stopped by addition of about 200 mL H₂O and the mixture is extracted with multiple portions of about 100 mL ethyl acetate, which is then washed with H₂O, then with multiple portions of about 25 mL of 10% aqueous NaHCO₃, and then with H₂O again. The ethyl acetate solution is dried over Na₂SO₄ and concentrated under reduced pressure. Further purification is by silica gel chromatography, developed with ethyl acetate: n-hexane, 1:1, v/v.

¹H NNR (500 MHZ, CDCl₃) δ 14.46 (s, 1H, 25 OH), 8.37 (d, 1H, H29, J=9.00 Hz, H29, H28), 8.03 (d, 2H, 30-(C═O)—NH₂), 7.08 (d, 1H, H2, J=8.5 Hz, H2, H1), 6.60 (d, 1H, H1, J=8.5 Hz, H1, H2), 6.53 (s, 1H, H4), 6.36 (d, 1H, H28, J=9.00 Hz, H28, H29), 5.97 (s, 1H, 23 NH), 5.56 (s, 1H, 3 OH), 4.69 (t, 1H, H17), 3.90 (s, 3H, 31-OCH₃), 2.77-2.70 (m, 6H, CH₂CH₂, 20, CH₂-21) 2.18-1.22 (m, 10H, remaining CH₂, ie 7, 15, 16, 11, 12), 0.76 (s, 3H, 18 CH₃).

### EXAMPLE 2

The following scheme depicts the synthetic route described in the following example.

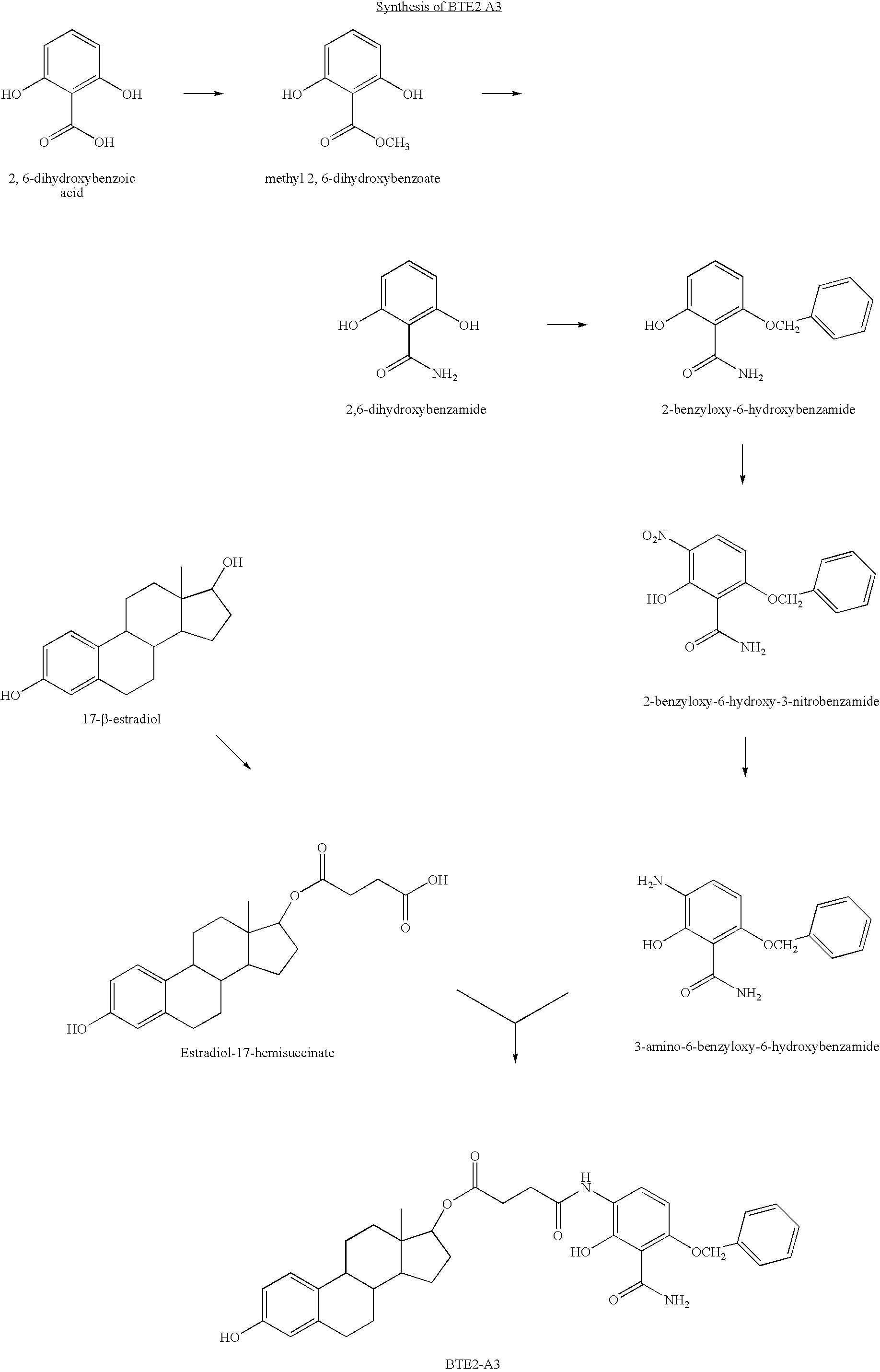

### A. 2,6-Dihydroxybenzoic Acid

2,6-Dihydroxybenzoic acid (CAS [303-07-1]) is purchased from Aldrich Chemical Company and is recrystallized from hot water then dried in a vacuum oven before use.

### B. Methyl 2,6-Dihydroxybenzoate

This is prepared in accordance with the procedure in Example 1.

### C. 2,6-Dihydroxybenzamide

This is prepared in accordance with the procedure in Example 1.

### D. 6-benzyloxy-2-hydroxybenzamide

In a round bottomed flask, about 24 g of 2,6 dihydroxybenzamide is dissolved in about 500 mL of dry acetone. To this is added about 50 g K₂CO₃ and the resultant product is stirred for about 30 minutes at room temperature. Benzyl bromide (1.2 eq.) is added dropwise and the reaction mixture is heated to reflux and held there for about 15 hours. The mixture is cooled in an ice bath and then is filtered.

The filtered solid is washed with multiple portions of about 50 mL acetone. Acetone is then partially removed using a rotary evaporator. The product is filtered and washed with water then recrystallized from benzene. The melting point is about 179° to 181° C.

¹H NMR (500 MHZ, DMSO-d₆) δ 14.01 (s, 1H, 2-OH), 8.13 (d, 2H, NH₂), 8.16-7.34 (m, 5H, C₆H₅), 7.30 (t, 1H, H4, J=8.5 Hz, H4, H3 and/or H5), 6.63 (d, 1H, H5, J=8.5 hz, h5, h4), 6.50 (d, 1H, H5, J=8.5 Hz, H3, H4), 5.28 (s, 2H, CH₂).

¹³C NMR (500 MHZ, DMSO-d₆) δ 171.72 (C═O), 163.75 (C2), 157.82 (C6), 136.24 (quaternary carbon of C₆H₅), 133.69 (C5), 128.73, 128.33, 127.97 (remaining carbons of C₆H₅), 110.61 (C3), 104.01 (C1), 103.03 (C4), 70.43 (CH₂).

### E. 6-benzyloxy-2-hydroxy-3-nitrobenzamide

6-benzyloxy 2-hydroxybenzamide (about 10 g) is dissolved in about 100 mL of glacial acetic acid in a round bottom flask. This solution is cooled in an ice bath and concentrated. About 12 ML of 0.2 M HNO₃ is added in multiple portions over a period of about 30 minutes. The mixture is stirred for about 6 hours at about 0 to 5° C., then for about 18 additional hours at room temperature.

The reaction mixture is diluted with about 1 L of cold water and the resulting solid is recovered by filtration. The filtrate is washed with about 500 mL of H₂O and multiple portions of about 50 mL ethanol. The product is dried in a vacuum desiccator over P₂O₅.

¹H NMR (500 MHZ, DMSO-d₆) δ 15.03 (s, 1H, 2-OH), 8.30 (d, 2H, NH₂), 8.10 (d, 1H, H₄, J=10 Hz, H4, H5) 7.50-7.35 (m, 5H, H—C₆H₅), 6.82 (d, 1H, H5, J=10 Hz, H5, H4), 5.43 (s, 2H, CH₂).

¹³C NMR (500 MHZ, DMSO-d₆) δ 169.86 (C=0), 161.77 (C3), 158.04 (C2) 135.56 (6C), 131.38 (quaternary carbon of C₆H₆), 130.10 (C4), 128.74, 128.55, 127.95, 127.80 (remaining carbons of C₆H₅), 107.16 (C1), 103.54 (C5), 71.00 (CH₂)

### F. 3 Amino 6-benzyloxy-2-hydroxybenzamide

About 7.3 g of 6-benzyloxy-2-hydroxy-3-nitrobenzamide is slurried in about 100 mL of dry CH₃OH, and 1.6 g charcoal. To this is added about 0.6 g FeCl₃.6H₂O and about 6 mL NH₂NH₂H₂O. The mixture is heated to reflux and held for about 24 hours. The resulting solution is concentrated by rotary evaporation, then filtered using Celite and concentrated under vacuum at about 35 to 40°. Product is extracted using ethyl acetate and the solution is concentrated using rotary evaporation.

¹H NMR (500 MHZ, DMSO-d₆) δ 14.32 (s, 1H, 2-OH) 8.13 (d, 2H, NH₂), 7.50-7.30 (m, 5H, H—C₆H₅), 6.81 (d, 1H, H5, J=8.5 Hz, H5, H4), 6.50 (d, 1H, H4, J=8.5 Hz, H4, H5), 5.16 (s, 2H, CH₂, 4.39 (s, 2H, 3-NH₂)

¹³C NMR (500 MHZ, DMSO-d₆) δ 172.41 (C═O), 151.16 (C2), 148.38 (C6), 136.64 (C3), 131.65 (quaternary carbon of C₆H₅, 128.63, 128.17, 127.98, 127.77 (remaining carbons of C₆H₅), 116.42 (C5), 103.06 (C1), 102.41 (C4), 70.54 (—CH₂).

### G. Estradiol-17-hemisuccinate

This compound is prepared in accordance with the procedure of Example 1.

### H. BTE2-A3

About 4 grams of estradiol-17-O-hemisuccinate is dissolved in about 25 mL dimethylformanide (DMF) and about 1.5 g of N-hydroxybenzotriazole (HOBT) is added. The mixture is cooled to about 0 to 5° C. and is stirred for about 30 minutes. Diisopropylcarbodiimide (about 1.5 g) is added, followed by stirring for about 30 minutes at about 0 to 5° C. A solution of about 2 grams of 3-amino-6-benzyloxy-2-hydroxybenzamide in about 10 mL DMF is added and the mixture is stirred for about 24 hours at room temperature. DMF is partially removed using rotary evaporation and is then redissolved in ethyl acetate. This is washed with multiple portions of about 25 mL 1N HCl, then H₂O, then multiple portions of 25 mL 10% aqueous NaHCO₃, then with H₂O again. The ethyl acetate solution is dried over Na₂SO₄ and concentrated under reduced pressure. Further purification is by silica gel chromatography, developed with ethyl acetate: n-hexane, 1:1, v/v.

¹H NMR (500 MHZ, CDCl₃) δ 14.46 (s, 1H, 25-OH), 8.40 (d, 1H, H29, J=9.00 Hz, H29, H28), 8.04(d, 2H, 30 C═ONH₂), 7.41-7.38 (C₆H₅), 7.09 (d, 1H, H2, J=8.5 Hz, H2, H1), 6.55 (d, 1H, H1 J=8.5 Hz, H1, H2), 6.53 (s, 1H, H4), 6.46 (d, 1H, H28, J=9.00 Hz; H28, H29), 5.97 (s, 1H, 23-NH), 5.09 (s, 2H, 31-CH₂), 4.70 (t,1H, H17), 2.77-2.70 (m,_{—}6H, CH₂-6, CH₂-20, CH₂-21), 2.20-1.22 (m, 10H, remaining CH₂ i.e., 7, 15, 16, 11, 12), 0.77 (s, 3H, 18-CH₃).

### EXAMPLE 3

The following scheme depicts the synthetic route for preparing the title compound as described below:

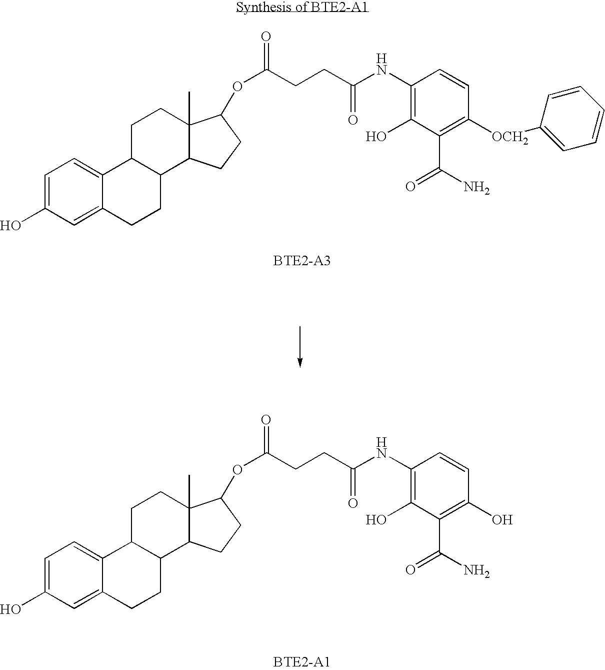

About 4 grams of BTE2-A3 are placed in solution with about 50 mL CH₃OH and 50 mL ethyl acetate. To this is added about 2 g Pd(C) 10% and then the slurry is placed under about 40 p.s.i. of H₂ for about 24 hours. The resultant product is filtered using Celite, then purified using silica gel chromatography, developed with ethyl acetate: n-hexane 1:1 (v/v), R_{f} is about 0.27, Yield is about 2.56 g.

### EXAMPLE 4

Preparation of

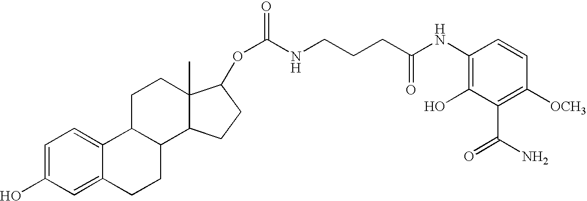

3-amino-2-hydroxy-6-methoxybenzamide is prepared as described above in Example 1. It is reacted with γ-butyrolactam under amide forming conditions to form the corresponding amide, (i.e., 3-(4-aminobutyrylamino)-2-hydroxy-6-methoxybenzamide).

Estradiol is reacted with 3-benzyl bromide to form the 3-benzyl protected derivative, which is reacted with phosgene to form the corresponding acid chloride. The acid chloride is reacted with the 3-(4-aminobutyrylamino)-2-hydroxy-6-methoxybenzamide product above. Hydrogenation thereof produces the above identified product.

### EXAMPLE 5

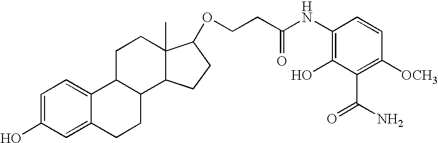

17-β-estradiol is reacted with benzyl bromide to form the corresponding benzyl protected derivative. The product is dissolved in acetone and mixed, at about 0 to 5° C., with chromic oxide to form the corresponding ketone. 1,3-propanediol and p-toluenesulfonic acid are reacted with the ketone to form the corresponding ketal. The ketal is reacted with lithium aluminum hydride and aluminum chloride in THF to form the corresponding 17-(3-hydroxypropoxy) derivative. Oxidation with chromium oxide yields the corresponding acid. The acid is reacted with 3-amino-2-hydroxy-6-methoxybenzamide, which is prepared as described in Example 1, followed by hydrogenation to form the above identified product.

### EXAMPLE 6

Preparation of

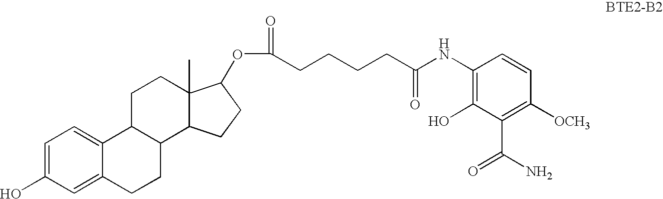

17β-estradiol is reacted with benzyl bromide to form the 3-benzyloxy derivative. This product is dissolved in acetone, reacted with adipic acid in the presence of p-toluene sulfonic acid under reflux to form the corresponding ester. The ester is reacted with 3-amino-2-hydroxy-6-methexybenzamide in the presence of dicyclohexylcarbondiimide followed by hydrogenation to form the above identified product.

### EXAMPLE 7

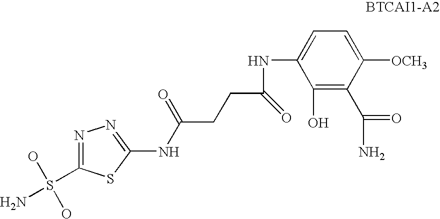

2-amino-1,3,4-thiadiazole-5-sulfonamide is reacted with succinic anhydride to form the succinamide derivative, 2-(4-carboxypropionylamino) 1,3,4-thiadiazole-5-sulfonamide. This product is reacted with 3-amino-2-hydroxy-6-methoxybenzamide in the presence of diisopropylcarbodiimide to form the above-identified compound.

### EXAMPLE 8

The procedure of Example 7 is followed except that 6-hydroxybenzothiazole-2-sulfonamide is used instead of 2-amino-1,3,4-thiadiazole-5-sulfonamide to form the product having the formula:

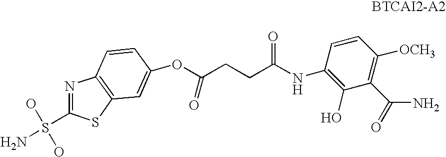

### EXAMPLE 9

The procedure of Example 8 is followed except that 3-amino-6-benzyloxy-2-hydroxybenzamide is used instead of the 3-amino-2-hydroxy-6-methoxybenzamide to form the corresponding product having the formula:

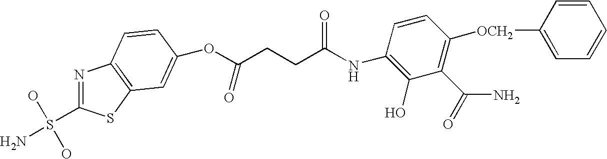

### EXAMPLE 10

The procedure of Example 7 is followed except that 3-amino-6-benzyloxy-2-hydroxybenzamide is used instead of 3-amino-2-hydroxy-6-methoxybenzamide to form the corresponding product having the formula:

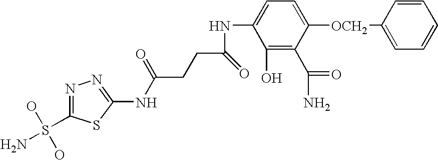

### EXAMPLE 11

The targeting of simvastatin, structure 1 in the scheme below, and Lovastatin, structure 2 in the scheme below, collectively referred to as the “statins,” to bone is accomplished by attaching the bone targeting agent to a non-critical (i.e., variable) part of the statin using a flexible linker as shown in the following scheme:

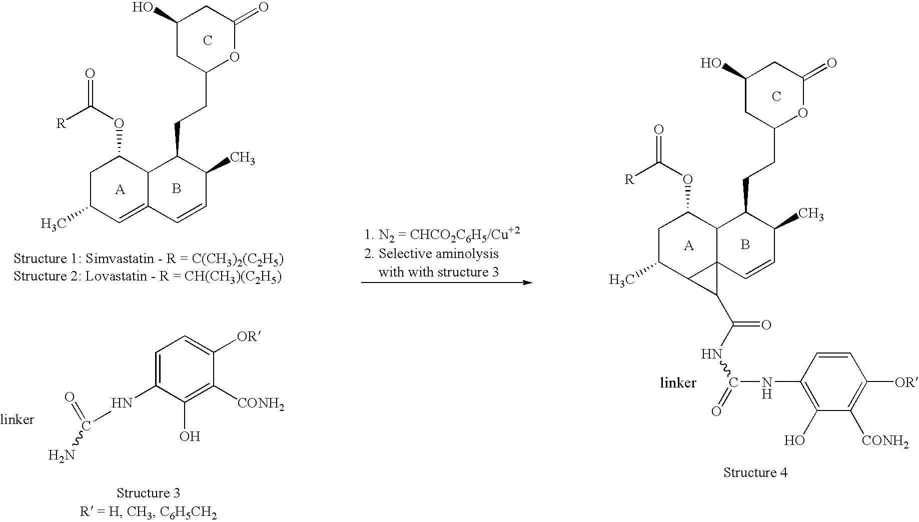

The least hindered face of the double bonds of the statins is the top face of the double bond of ring A. Using a prodrug of the statins having the active beta-hydroxy acid function lactonized, as shown in ring C, the double bond of ring A is cyclopropanated with phenyl diazoacetate using standard procedures. The resulting cyclopropane carboxylic acid phenyl ester is then subjected to selective aminolysis employing a molecule such as structure 3, shown in the scheme above. By using the phenyl ester, the reactivity of that ester carbonyl group is raised multi-fold above those of the competing lactone and ester side chain moieties. The final bone-targeted statin would have structure 4, shown in the scheme above.

In Vitro Testing of Bone Targeting

The ability of the compounds of the present invention to act as bone targeted pharmaceuticals is estimated initially by determination of the ability of the compounds to be bound to microcrystalline hydroxyapatite [Ca₁₀(PO₄)6.OH₂] (HA) from a dilute aqueous solution.

Solutions of test compounds are constructed in about 99: 1, v/v, H₂O:dimethylsulfoxide (DMSO) at 10⁻⁵M. These solutions are taken for determination of electronic photometric absorption spectra scanning from λ=500-190 nm. Absorption maxima (λₘₐₓ) and extinction coefficients (ε) are determined using the Beer-Lambert law. The data is provided in Table C.

TABLE C

| Compound | λₘₐₓ (nm) | ∈ (M⁻¹ × cm⁻¹) |
|---|---|---|
| 2,6-dihydroxy-3-nitrobenzoic acid | 340 | 25,230 |
| 2,6-dihydroxy-3-nitrobenzamide | 395 | 9,220 |
| 17,β-estradiol | 278 | 2,620 |
| 3-amino-2,6-dihydroxybenzamide | 338 | 2,050 |
| Tetracycline | 362 | 12,880 |
| BTE2-A1 | 330 | 2,380 |
| BTE2-A2 | 338 | 5,100 |
| BTE2-B2 | 338 | 4,160 |

For binding determinations, 1 mL of each solution is taken and added to 0.1 mL of trishydroxymethylaminomethane (50 mM) in 1% DMSO (aq) that contains either 0 or 0.5% (w/v) of slurried HA. These solutions and slurries are mixed for about 4 minutes, then centrifuged for about 3 minutes at 10,000×g. Supernatants are taken for UV absorption spectrometry at previously determined λₘₐₓ. Concentrations of test compound are determined and the extent of binding is calculated. Tetracycline is included as a positive control compound. The results are set forth in Table D.

TABLE D
Binding to Hydroxyapatite

| Compound | % Adsorbed | Binding Index |
|---|---|---|
| 17-β-estradiol | −4.0% | −8 |
| 3-amino-2,6-dihydroxybenzamide | 25.4% | 49 |
| Tetracycline | 51.6% | 100 |
| BTE2-A1 | 54% | 105 |
| BTE2-A2 | 91.7% | 178 |
| BTE2-B2 | 99.1% | 192 |
| BTE2-C2 | 100% | >200 |
| BTE2-D1 | 92% | 198 |
| BTE2-D2 | 93% | 198 |
| BTE2-D3 | 94% | 201 |
| BTE2-E2 | 100% | >200 |

The data in Table D show that the compounds prepared in the present invention have a strong affinity for hydroxyapatite.

In Vivo Testing of Efficacy

In vivo experiments—Bilaterally ovariectomized and sham operated Sprague-Dawley female rats (150-170 g) from Harlan Laboratories (Indianapolis, Ind.) are housed on about a 12 hour dark/light cycle at 22° C. with ad libitum access to tap water and food (Purina Laboratory Rodent Diet 5001: 0.95% calcium, 0.67% total phosphorous, and 0.40% non-phytate phosphorous). The rats are randomly assigned into groups of about 6 to 10 animals.

Compound administration begins about 4 days after surgery. Injections are given in the scruff of the neck or by gavage about 3 times a week (e.g., Monday, Wednesday, and Friday) for about 6 weeks in a volume of about 0.2 mL/kg body mass for subcutaneous experiments or about 0.5 mL/kg body mass for oral experiments. All compounds are dissolved in 5% DMSO in corn oil.

After about 6 weeks of dosing, animals are euthanized by carbon dioxide asphyxiation. Blood is collected by cardiac puncture, and uteri and both left and right femurs are removed. Fresh weights of uteri are obtained. All animal procedures and care are approved by the internal animal care and use committee to ensure compliance with NIH guidelines.

Determination of whole femoral density.—Following removal, femurs are rubbed clean of soft tissue using gauze, and femur fresh weights are measured. Femurs are then submerged in distilled water under a vacuum for about 1 hour to completely hydrate the femurs. Densities are determined by Archimedes' Principle using the following formula: density=[mass of hydrated femur/(mass of hydrated femur-mass of hydrated femur submerged in water)]*density of water at ambient temperature.

The dose that is effective in preventing about 50% of the bone loss associated with OVX (ED50) is determined by interpolation. The data is set forth in Table E.

TABLE E

| | ED50 (nmol/kg) |
|---|---|
| | for preservation |
| Compound | of Femoral Mass |
| BTE₂-A1 | 8 |
| BTE₂-A2 | 19 |
| BTE₂-B2 | 9 |
| BTE₂-C2 | 215 |
| BTE₂-D2 | 59 |
| BTE₂-D3 | 21 |
| BTE₂-E2 | 38 |
| Estradiol | 10 |
| Raloxifene | 210 |

Quantitative Computed Tomography (QCT) analysis.—Each femur is wrapped in gauze, soaked in about 0.9% saline solution, and frozen at about −20° C. until analysis. Femurs are thawed at room temperature before they are scanned. The pQCT scans are performed on a Stratech XCT RM pQCT machine (Norland, Fort Atkinson, Wis.). A total length measurement of each femur is obtained. A preliminary view is used to determine the reference point before a CT scan (0.5 mm slice) is performed on the distal end of the femur at a distance of 18% of the total femur length. The analyses of the trabecular bone mineral content and density are performed using manufacturer's small animal software. The coefficients of variation of each parameter are less than about 5% based on about 5 consecutive determinations of the same bone with repositioning between each measurement.

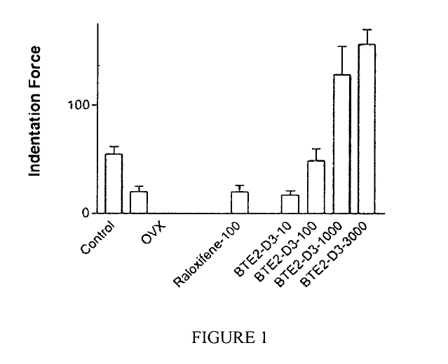

Studies with BTE₂-D3—QCT data are obtained that indicated a density of bone greater than that of control or estradiol treated animals. Bone density is not necessarily the equivalent to bone strength, so an additional test (blunt indentation force of the distal metaphysis) is performed as well. With reference to FIG. 1, as a measure of bone mechanical competence, a blunt indentation force to failure test is performed on femora from about 6-week-old OVX rats. Sham operated animals are simultaneously tested as a control. The exemplary compound of the present invention, BTE₂-D3 shows increased bone strength in a dose dependent manner. Raloxifene, at a normal dose, has a small (e.g., 30%) effect. Estradiol is able to completely protect against osteopenia. These data show that BTE₂-D3 has a greater maximum effect on bone density than does the parent estradiol.

Other embodiments of the invention will be apparent to those skilled in the art from consideration of the specification and practice of the invention disclosed in this application. It is intended that the Specification and Example be considered as exemplary only, and not intended to limit the scope and spirit of the invention.

Unless otherwise indicated, all numbers expressing quantities of ingredients, properties such as reaction conditions, and so forth used in the Specification and claims are to be understood as being modified in all instances by the term “about.” Accordingly, unless indicated to the contrary, the numerical parameters set forth are approximations that may vary depending upon the desired properties sought to be determined by the present invention.

Notwithstanding that the numerical ranges and parameters setting forth the broad scope of the invention are approximations, the numerical values set forth in the examples are reported as precisely as possible. Any numerical value, however, inherently contain certain errors necessarily resulting from the standard deviation found in their respective testing measurements.

Throughout this application, various publications are referenced. All such references are incorporated herein by reference.

1. A compound of the formula: 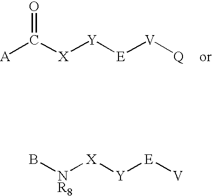
or pharmaceutically acceptable salts thereof wherein
A and B are each a bone targeting moiety;
X is an alkylene group containing from 1-10 carbon atoms on the main chain and up to a total of 20 carbon atoms; 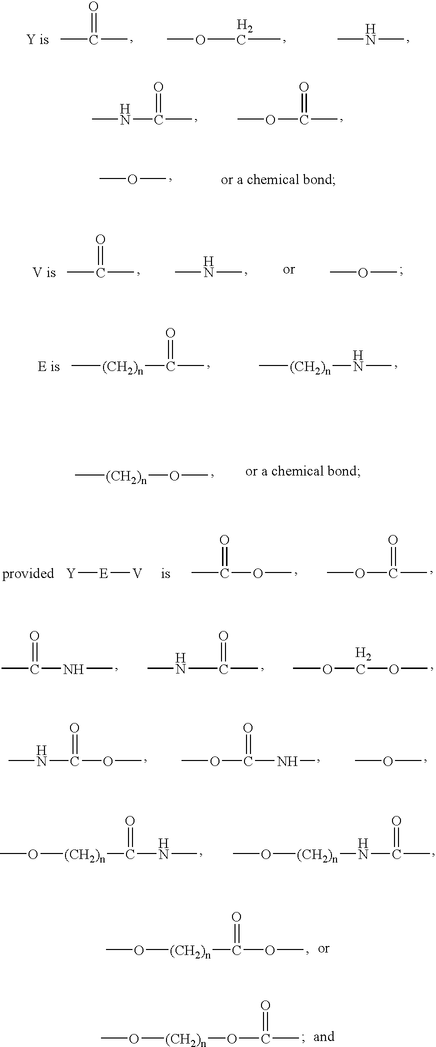
Q is an agent which interacts with the bone and affects bone metabolism by inhibiting bone resorption, increasing bone formation, or both, Q having a hydroxy group, an amino group or oxo group before being bonded to V, said hydroxy group, amino group, oxo group, whichever is present, being eliminated from said agent when bonded to V; and n is an integer from 0 to 6.

2. The compound according to claim 1 wherein the bone targeting moiety A is: 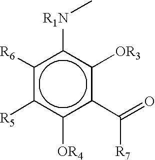
the bone targeting moiety B is: 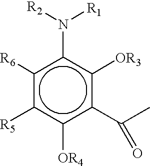
wherein
R₁ is hydrogen, lower alkyl or aryl lower alkyl;
R₂ is hydrogen, lower alkyl or aryl lower alkyl;
R₃ is hydrogen or lower alkyl;
R₄ is hydrogen, aryl, aryl lower alkyl, or lower alkyl;
R₅ and R₆ are independently hydrogen or lower alkyl or R₅ and R₆ taken together with the carbon atoms to which they are bonded form a ring containing 4 to 10 ring carbon atoms and up to a total of 18 carbon atoms;
R₇ is hydroxy, lower alkoxy, or NR₈R₉;
R₈ is hydrogen or lower alkyl; and
R₉ is hydrogen or lower alkyl.

3. The compound according to claim 1 wherein Q is a carbonic anhydrase inhibitor, a sex hormone, Vitamin D, or DHEA.

4. The compound according to claim 1 wherein is R₇ is NR₈R₉.

5. The compound according to claim 1 wherein R₃ is hydrogen.

6. The compound according to claim 1 wherein R₅ and R₆ are independently hydrogen or lower alkyl.

7. The compound according to claim 1 wherein X is an alkylene bridge containing 2-6 carbon atoms.

8. The compound according to claim 7 wherein X contains 2-4 carbon atoms.

9. The compound according to claim 1

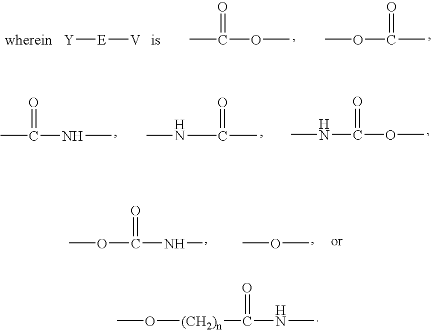

10. The compound according to claim 1 wherein the carbonic anhydrase inhibitor is 2-amino-1-3,4-thiadiazole-5-sulfonamide or 5-hydroxybenzothiazole sulfonamide; the androgenic agent is testosterone or androstenedione; and the estrogenic agent is estriol or estradiol.

11. The compound according to claim 1 of the formula 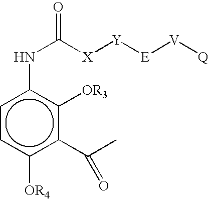
wherein
R₃ is hydrogen or lower alkyl;
R₄ is hydrogen, lower alkyl, aryl or lower arylalkyl;
X is an alkylene group containing 1-6 carbon atoms; 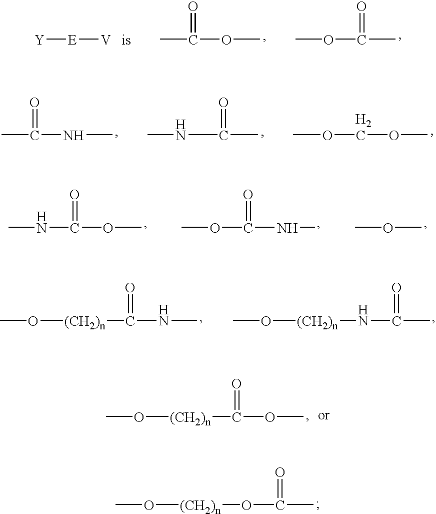
Q is a carbonic anhydrase inhibitor, a sex hormone, Vitamin D or DHEA, all of which are less a hydroxy group, an amino group or oxo group, whichever is present, at the carbon atom bonded to V; and
n is an integer from 0 to 6.

12. The compound according to claim 11 wherein the estrogenic agent is estriol or estradiol; the androgenic agent is testosterone or androstenedione; and the carbonic anhydrase inhibitor is 2-amino-1,3,4-thiadiazole-5-sulfonamide or 5-hydroxybenzothiazole sulfonamide.

13. The compound according to claim 11 which is

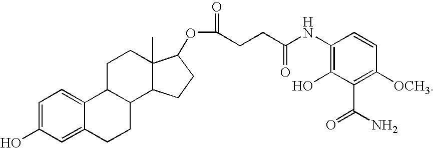

14. The compound according to claim II which is

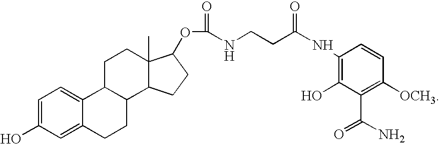

15. The compound according to claim II which is

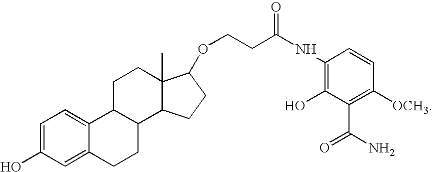

16. A pharmaceutical composition comprising a pharmaceutically effective amount of a compound according to claim 1 or claim 11 and a pharmaceutically acceptable carrier therefor.

17. A method for the treatment or prophylaxis of degenerative bone disorders in an animal in need of such treatment which comprises administering thereto an effective amount of a compound according to claim 1 or claim 11.

18. The method according to claim 17 wherein said animal is a mammal.

19. The method according to claim 17 wherein the degenerative bone disease is osteoporosis.
